(19) United States
(12) Patent Application Publication
(10) Pub. No.: US 2021/0169348 A1
(43) Pub. Date: Jun. 10, 2021

(54) PHYSIOLOGICAL INFORMATION PROCESSING APPARATUS, PHYSIOLOGICAL INFORMATION PROCESSING METHOD, PROGRAM AND STORAGE MEDIUM

(71) Applicant: NIHON KOHDEN CORPORATION, Tokyo (JP)

(72) Inventors: Mami SAKAI, Tokorozawa-shi, Saitama (JP); Yoshihiro SUGO, Tokorozawa-shi, Saitama (JP)

(73) Assignee: NIHON KOHDEN CORPORATION, Tokyo (JP)

(21) Appl. No.: 17/263,749

(22) PCT Filed: Aug. 23, 2019
(86) PCT No.: PCT/JP2019/033120

§ 371 Date: Jan. 27, 2021

(30) Foreign Application Priority Data
Sep. 6, 2018 (JP) 2018-166764

(51) Int. Cl. A61B 5/0205; A61B 5/352

(52) U.S. Cl. CPC A61B 5/0205; A61B 5/0816; A61B 5/352

(57) ABSTRACT

A method includes: acquiring electrocardiogram data of a subject; acquiring pulse wave data of the subject; calculating RR intervals in a predetermined time interval based on the electro-cardiogram data; calculating pulse wave transit times (PWTT) in the predetermined time interval based on the electrocardiogram data and the pulse wave data; calculating a pulse wave transit time variation (PWTTV) in the predetermined time interval based on the PWTT in the predetermined time interval; determining whether the PWTTV in the predetermined time interval satisfies a predetermined condition associated with previously calculated PWTTV or not; calculating corrected values (PWTT′) of the PWTT based on the PWTT and the RR intervals in a case where the PWTTV does not satisfy the predetermined condition; and determining candidate values (PWTT) of the PWTT based on the PWTT and PWTT′.

### TECHNICAL FIELD

The present disclosure relates a physiological information processing apparatus and a physiological information processing method. Further, the present disclosure relates to a program for causing a computer to execute the physiological information processing method, and a computer-readable storage medium having the program stored therein.

### BACKGROUND ART

JP-T-2015-519940 discloses a method for determining a cardiac output of a patient from one or more physiological characteristics of the patient. Particularly, JP-T-2015-519940 discloses a method for determining the cardiac output based on a pulse wave transit time (PWTT) which is a time interval between a peak point of an R wave and a rise point of a pulse waveform appearing due to the R wave.

A pulse wave transit time (which will be hereinafter abbreviated to PWTT) may be unable to be identified accurately when an RR interval indicating a time interval between adjacent R waves is short. With respect to this point, when another R wave is present between a predetermined R wave and a pulse waveform appearing due to the predetermined R wave, a time interval between a peak point of the other R wave and a rise point of the pulse waveform is mistakenly identified as a PWTT in a background-art PWTT calculation process. Thus, there is a possibility that the PWTT cannot be identified correctly when the PWTT is longer than the RR interval. In such a case, calculation accuracy of the PWTT is lowered. Accordingly, accuracy of physiological information such as blood pressure, a cardiac output, etc. of a patient calculated based on the PWTT is lowered. From the aforementioned viewpoint, there is still room for further improving the calculation accuracy of the PWTT.

### SUMMARY

The present disclosure provides a physiological information processing method and a physiological information processing apparatus which can further improve calculation accuracy of a PWTT. In addition, the present disclosure provides a physiological information processing method, a program for causing a computer to execute the physiological information processing method, and a computer-readable storage medium having the program stored therein.

According to one or more aspects of the present disclosure, there is provided a physiological information processing method executed by a computer.

The method comprises:

acquiring electrocardiogram data of a subject;

acquiring pulse wave data of the subject;

calculating a plurality of RR intervals in a predetermined time interval based on the electrocardiogram data;

calculating a plurality of pulse wave transit times (PWTT) in the predetermined time interval based on the electrocardiogram data and the pulse wave data;

calculating a pulse wave transit time variation (PWTTV) in the predetermined time interval based on the plurality of PWTT in the predetermined time interval;

determining whether the PWTTV in the predetermined time interval satisfies a predetermined condition associated with a plurality of previously calculated PWTTV or not;

calculating corrected values (PWTT′) of the plurality of PWTT based on the plurality of PWTT and the plurality of RR intervals in a case where the PWTTV does not satisfy the predetermined condition; and

determining candidate values (PWTT_{c}) of the plurality of PWTT based on the plurality of PWTT and the plurality of PWTT′.

According to one or more aspects of the present disclosure, there is provided a physiological information processing apparatus.

The physiological information processing apparatus comprises: at least one processor; and a memory storing a computer-readable instruction. When executed by the at least one processor, the computer-readable instruction causes the physiological information processing apparatus to perform operations comprising:

acquiring electrocardiogram data of a subject;

acquiring pulse wave data of the subject;

calculating a plurality of RR intervals in a predetermined time interval based on the electrocardiogram data;

calculating a plurality of pulse wave transit times (PWTT) in the predetermined time interval based on the electrocardiogram data and the pulse wave data;

calculating a pulse wave transit time variation (PWTTV) in the predetermined time interval based on the plurality of PWTT in the predetermined time interval;

determining whether the PWTTV in the predetermined time interval satisfies a predetermined condition associated with a plurality of previously calculated PWTTV or not;

calculating corrected values (PWTT′) of the plurality of PWTT based on the plurality of PWTT and the plurality of RR intervals in a case where the PWTTV does not satisfy the predetermined condition; and

determining candidate values (PWTT_{c}) of the plurality of PWTT based on the plurality of PWTT and the plurality of PWTT′.

### BRIEF DESCRIPTION OF DRAWINGS

FIG. 1 illustrates an example of a hardware configuration of a physiological information processing apparatus according to an embodiment of the present invention.

FIG. 2 illustrates a flow chart for explaining an example of a physiological information processing method according to the embodiment of the present invention.

FIG. 3 illustrates a flow chart for explaining an example of a process for calculating a PWTT variation (PWTTV).

FIG. 4 illustrates an example of an electrocardiogram (ECG) waveform and a pulse waveform for explaining a corrected value (PWTT′) of a PWTT.

FIG. 5 illustrates a flow chart for explaining an example of a process for determining each of candidate values (PWTT_{c}) of a plurality of PWTT.

FIG. 6 illustrates a flow chart for explaining an example of a process for calculating a PWTT_{c} variation (PWTTV_{c}).

### DESCRIPTION OF EMBODIMENTS

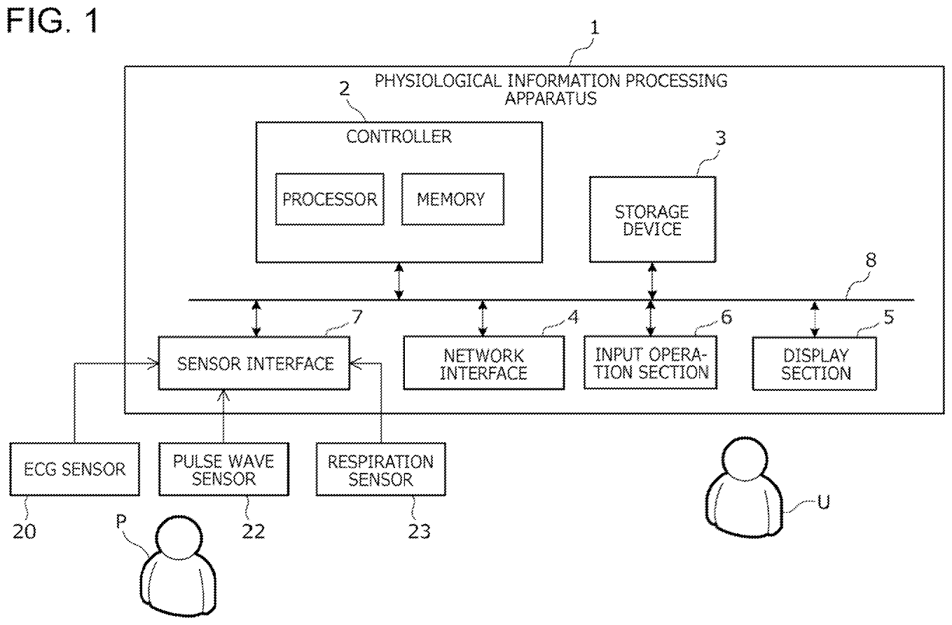

An embodiment of the present invention will be described below with reference to the drawings. First, a hardware configuration of a physiological information processing apparatus 1 according to the embodiment of the present invention (which will be hereinafter referred to as present embodiment simply) will be described below with reference to FIG. 1.

FIG. 1 is a diagram showing an example of the hardware configuration of the physiological information processing apparatus 1 according to the present embodiment. As shown in FIG. 1, the physiological information processing apparatus 1 (which will be hereinafter referred to as processing apparatus 1 simply) includes a controller 2, a storage device 3, a network interface 4, a display section 5, an input operation section 6, and a sensor interface 7, which are connected communicably with one another through a bus 8.

The processing apparatus 1 may be a dedicated apparatus (such as a patient monitor etc.) for displaying a trend graph of vital signs of a subject P. In addition, the processing apparatus 1 may be a personal computer, a work station, a smartphone, a tablet, or a wearable device (such as a smart watch, AR glasses, or the like) worn on the body (such as an arm, the head, or the like) of a medical worker U.

The controller 2 includes at least one memory and at least one processor. The memory is configured to store computer-readable commands (programs). For example, the memory may be constituted by an ROM (Read Only Memory) where the various programs etc. are stored, an RAM (Random Access Memory) having work areas where the various programs etc. to be executed by the processor are stored, etc. In addition, the memory may be constituted by a flash memory etc. The processor may be, for example, a CPU (Central Processing Unit), an MPU (Micro Processing Unit), and/or a GPU (Graphics Processing Unit). The CPU may be constituted by a plurality of CPU cores. The GPU may be constituted by GPU cores. The processor may have a configuration in which the processor expands a program designated from the various programs incorporated into the storage device 3 or the ROM onto the RAM, and executes various processes in cooperation with the RAM.

The controller 2 may control various operations of the processing apparatus 1 when the processor expands a physiological information processing program which will be described later onto the RAM and executes the program in cooperation with the RAM. Details of the physiological information processing program will be described later.

The storage device 3 is such as an HDD (Hard Disk Drive), an SSD (Solid State Drive), a flash memory, or the like. The storage device 3 is configured to store programs or various data. The physiological information processing program may be incorporated into the storage device 3. In addition, physiological information data such as electrocardiogram (ECG) data, pulse wave data, respiration data, etc. of the subject P may be stored in the storage device 3. For example, ECG data acquired by an ECG sensor 20 may be stored in the storage device 3 through the sensor interface 7.

The network interface 4 is configured to connect the processing apparatus 1 to a communication network. Specifically, the network interface 4 may include various wired connection terminals for making communication with an external apparatus such as a server through the communication network. In addition, the network interface 4 may include various processing circuits and an antenna etc. for making wireless communication with an access point. A wireless communication standard between the access point and the processing apparatus 1 is, for example, Wi-Fi (registered trademark), Bluetooth (registered trademark), ZigBee (registered trademark), LPWA or a 5th Generation mobile communication system (5G). The communication network is an LAN (Local Area Network), a WAN (Wide Area Network), or the Internet etc. For example, the physiological information processing program or the physiological information data may be acquired through the network interface 4 from the server disposed on the communication network.

The display section 5 may be a display device such as a liquid crystal display, an organic EL display, or the like. In addition, the display section 5 may be a display device such as a transmissive type or a non-transmissive type head mount display, an AR display, or the like, worn on the head of an operator. Further, the display section 5 may be a projector device projecting images onto a screen.

The input operation section 6 is configured to accept an input operation from the medical worker U operating the processing apparatus 1, and create an instruction signal in response to the input operation. The input operation section 6 is, for example, a touch panel disposed to be superimposed on the display section 5, an operation button attached to a housing, a mouse and/or keyboard, or the like. After the instruction signal created by the input operation section 6 is transmitted to the controller 2 through the bus 8, the controller 2 executes a predetermined action in response to the instruction signal.

The sensor interface 7 is an interface for connecting vital sensors such as the ECG sensor 20, a pulse wave sensor 22, a respiration sensor 23, etc. communicably with the processing apparatus 1. The sensor interface 7 may include input terminals to which the physiological information data outputted from the vital sensors are inputted. The input terminals may be physically connected with connectors of the vital sensors. In addition, the sensor interface 7 may include various processing circuits and an antenna etc. for making wireless communication with the vital sensors.

The ECG sensor 20 is configured to acquire ECG data expressing an ECG waveform of the subject P. The pulse wave sensor 22 is configured to acquire pulse wave data expressing pulse waves of the subject P. The respiration sensor 23 is configured to acquire respiration waveform data expressing a respiration waveform of the subject P.

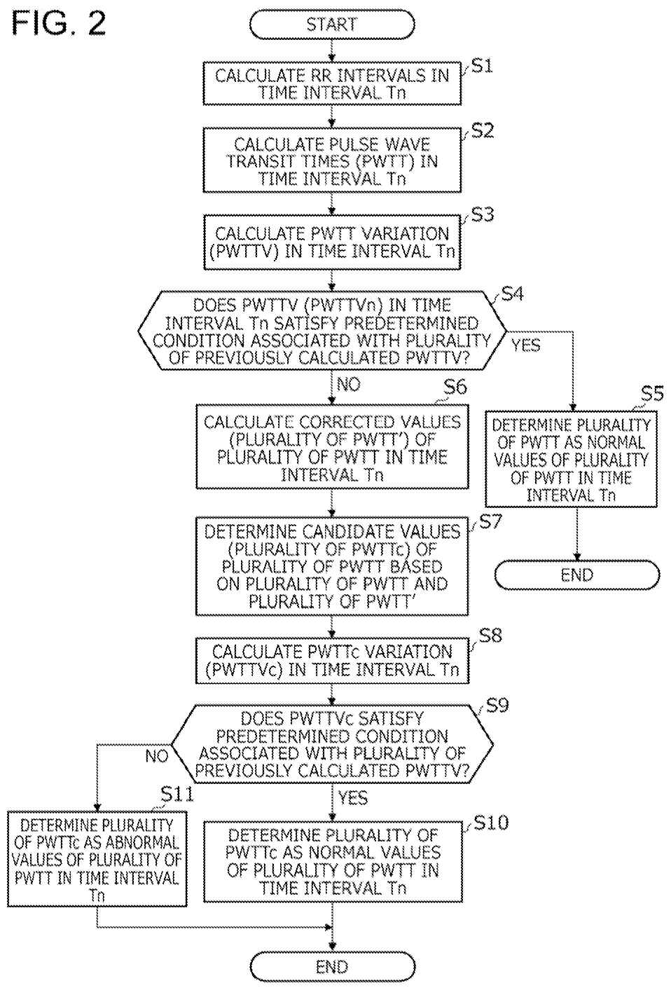

Next, a physiological information processing method according to the present embodiment will be described below with reference to FIG. 2. FIG. 2 is a flow chart for explaining an example of the physiological information processing method according to the present embodiment. As shown in FIG. 2, first, the controller 2 acquires ECG data of a subject P from the ECG sensor 20, and acquires pulse wave data of the subject P from the pulse wave sensor 22. Further, the controller 2 acquires respiration waveform data of the subject P from the respiration sensor 23.

Next, the controller 2 calculates a plurality of RR intervals in a time internal Tₙ (an example of a predetermined time interval, a is a natural number) based on the acquired ECG data and the acquired respiration waveform data (step S1). Here, each of the RR intervals means a time interval between peak points of adjacent ones of the R waves. For example, after a respiration interval (a time interval between inspiration and expiration) has been identified based on the respiration waveform data, the controller 2 may determine the identified respiration interval as the time interval Tₙ. Next, the controller 2 may calculate a plurality of RR intervals in the time interval Tₙ from the ECG data in the time interval T. Then, after a next identified respiration interval has been determined as a time interval Tₙ₊₁, the controller 2 may calculate a plurality of RR intervals in the time interval Tₙ₊₁.

Incidentally, although each respiration interval is identified based on the respiration waveform data acquired from the respiration sensor 23 in the present embodiment, the respiration interval may be identified from the ECG data or the pulse wave data. In this case, the respiration interval may be identified from the ECG waveform or an envelope of pulse waves. In addition, in the present embodiment, the respiration interval is determined as the time interval Tₙ. However, the time interval Tₙ may be determined beforehand. With respect to this point, the time interval may have a predetermined time width (e.g. 10 seconds). In addition, in the present embodiment, after a plurality of RR intervals have been first calculated, a plurality of RR intervals in the time interval Tₙ may be selected.

Next, in a step S2, the controller 2 calculates a plurality of pulse wave transit times (PWTT) in the time interval Tₙ based on the ECG data, the pulse wave data and the respiration waveform data. Here, each of the plurality of PWTT means a time interval between a peak point of a predetermined R wave in the ECG data and a rise point of a predetermined pulse waveform appearing due to the predetermined R wave. For example, after the time interval Tₙ has been identified from the respiration waveform data, the controller 2 may calculate the plurality of PWTT in the time interval Tₙ from the ECG data and the pulse wave data in the time interval Tₙ. In addition, as a calculation method of each of the plurality of PWTT, the controller 2 first identifies a time instant of the peak point of the predetermined R wave from the ECG data, and identifies a time instant of the rise point of the predetermined pulse waveform appearing next to the predetermined R wave from the pulse wave data. Next, the controller 2 calculates a time interval between the time instant of the rise point of the predetermined pulse waveform and the time instant of the peak point of the predetermined R wave to thereby measure the PWTT.

Incidentally, when the RR interval is shorter than the PWTT, it may be assumed that another R wave is present between the predetermined R wave and the pulse waveform appearing due to the predetermined R wave. In this case, a time interval between a peak point of the other R wave and the rise point of the pulse waveform is mistakenly identified as the PWTT. Thus, there is a fear that the PWTT cannot be calculated correctly in accordance with a length of the RR interval (in other words, in accordance with a heartbeat condition of the subject) by the calculation method of the PWTT in the step S2. Thus, in the physiological information processing method according to the present embodiment, in order to further improve calculation accuracy of the PWTT, it is determined whether the calculated PWTT is a normal value or not, and the calculated PWTT is corrected when the calculated PWTT is not the normal value. In addition, in the present embodiment, after a plurality of PWTT are first calculated, a plurality of PWTT in the time interval Tₙ may be selected.

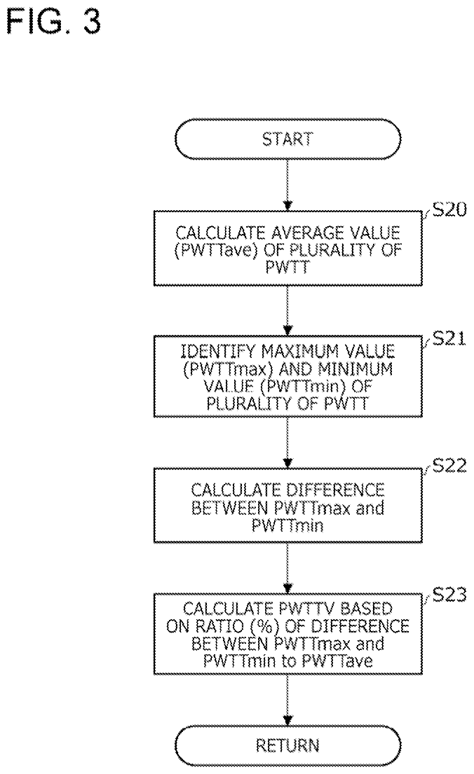

Next, in a step S3, the controller 2 calculates a PWTT variation PWTTV in the time interval Tₙ. Here, an example of a calculation method of the PWTTV will be described with reference to FIG. 3. FIG. 3 is a flow chart for explaining an example of a process for calculating the PWTTV.

As shown in FIG. 3, the controller 2 first calculates an average value PWTTₐᵥₑ of the plurality of PWTT in the time interval Tₙ (step S20). For example, the PWTTₐᵥₑ can be expressed by the following expression. Assume here that m PWTTi (i=1, 2, . . . m) are present in the time interval Tₙ.

$$\begin{matrix}[ Math . 1 ] & \\ {PWTT}_{a ν e} = \frac{1}{m} {\sum }_{i = 1}^{m} PWTTi & ( 1 )\end{matrix}$$

Next, the controller 2 identifies a maximum value PWTTₘₐₓ and a minimum value PWTTₘᵢₙ of the plurality of PWTT in the time interval respectively (step S21). Then, the controller 2 calculates a difference between the maximum value PWTTₘₐₓ, and the minimum value PWTTₘᵢₙ (step S22). Next, in a step S23, the controller 2 calculates the PWTTV based on a ratio (%) of the difference between the maximum value PWTTₘₐₓ and the minimum value PWTTₘᵢₙ to the average value PWTTₐᵥₑ of the plurality of PWTT. For example, the PWTTV can be expressed by the following expression. Thus, the PWTTV in the time interval Tₙ can be calculated.

[Math.2]

PWTTV=(PWTTₘₐₓ−PWTTₘᵢₙ)/PWTTₐᵥₑ×100% (2)

Return to FIG. 2. In a step S4, the controller 2 determines whether the PWTTV in the time interval Tₙ (which will be hereinafter denoted by PWTTVₙ) satisfies a predetermined condition associated with a plurality of previously calculated PWTTV or not. For example, a PWTTV in a time interval Tₙ₋₁ which is a time interval one time before the time interval Tₙ is denoted by PWTTVₙ₋₁, and PWTTV in a time interval Tₙ₋₂ which is a time interval two times before the time interval Tₙ is denoted by PWTTVₙ₋₂. In addition, a PWTTV in a time interval Tₙ₋ₚ which is a time interval p-times before the time interval Tₙ (p is a natural number equal to or larger than 3) is denoted by PWTTVₙ₋ₚ. In this case, the controller 2 first calculates an average value of the plurality of previously calculated PWTTV (i.e. an average value PWTTVₐᵥₑ of the PWTTVₙ₋₁ to the PWTTVₙ₋ₚ) based on the following expression. Incidentally, the value of p may be set suitably on the side of a medical facility.

$$\begin{matrix}[ Math . 3 ] & \\ P W T T {V}_{a ν e} = \frac{1}{p} {\sum }_{i = 1}^{p} {PWTTV}_{n - i} & ( 3 )\end{matrix}$$

Next, the controller 2 determines whether the PWTTVₙ is included in a predetermined range set based on the average value PWTTVₐᵥₑ or not. Specifically, the controller 2 determines whether the PWTTVₙ satisfies the following conditional expression or not. Here, α is a predetermined value which may be suitably set on the side of the medical facility. For example, α may be the predetermined value in a range of from 1% to 10%.

[Math.4]

PWTTVₐᵥₑ−α≤PWTTVₙ≤PWTTVₐᵥₑ+α (4)

Thus, it is determined whether the PWTTVₙ satisfies the predetermined condition which is relevant to the PWTTVₐᵥₑ and defined by the aforementioned expression (4) or not. When the determination of the step S4 results in YES, the controller 2 determines the plurality of calculated PWTT as normal values of the plurality of PWTT in the time interval Tₙ (step S5). In this case, the plurality of PWTT determined as the normal values are stored in the memory or the storage device 3. On the other hand, when the determination of the step S4 results in NO, the present process goes to a step S6.

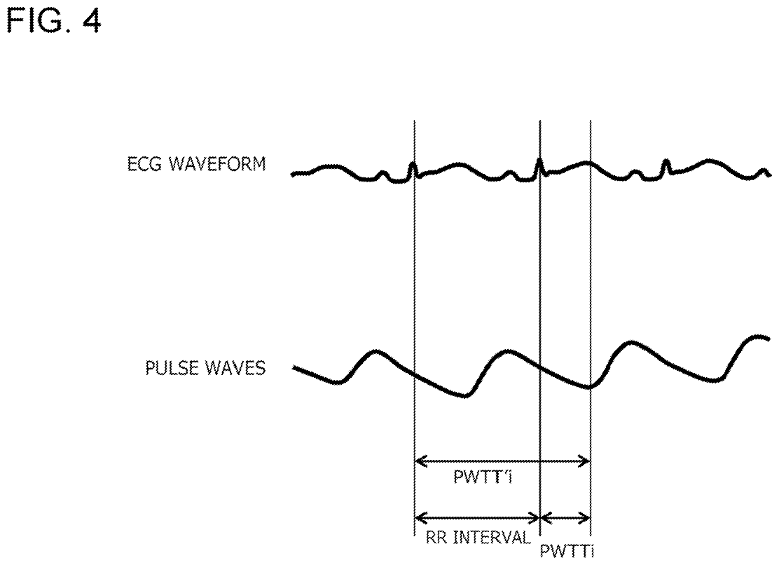

In the step S6, the controller 2 calculates a plurality of PWTT′ which are corrected values of the plurality of PWTT in the time interval Tₙ. With respect to this point, the controller 2 adds an RR interval calculated immediately before each PWTTᵢ (i=1, 2, . . . m), to the PWTTᵢ to thereby calculate a PWTT′ᵢ which is a corrected value of the PWTTᵢ, as shown in FIG. 4. For example, the relation between the PWTTᵢ and the PWTT′ᵢ can be expressed by the following expression.

[Math.5]

PWTT′ᵢ=PWTTᵢ+RR interval immediately previous thereto (5)

In some case, the PWTT may be unable to be calculated correctly when the RR interval is shorter than the PWTT, as described above. Therefore, the PWTT′ᵢ which is a time interval between a peak point of an R wave appearing immediately before an R wave associated with the PWTTᵢ and a rise point of a pulse waveform is determined as a corrected value of the PWTTᵢ. In this manner, m PWTT′ᵢ which are corrected values of m PWTTᵢ are calculated.

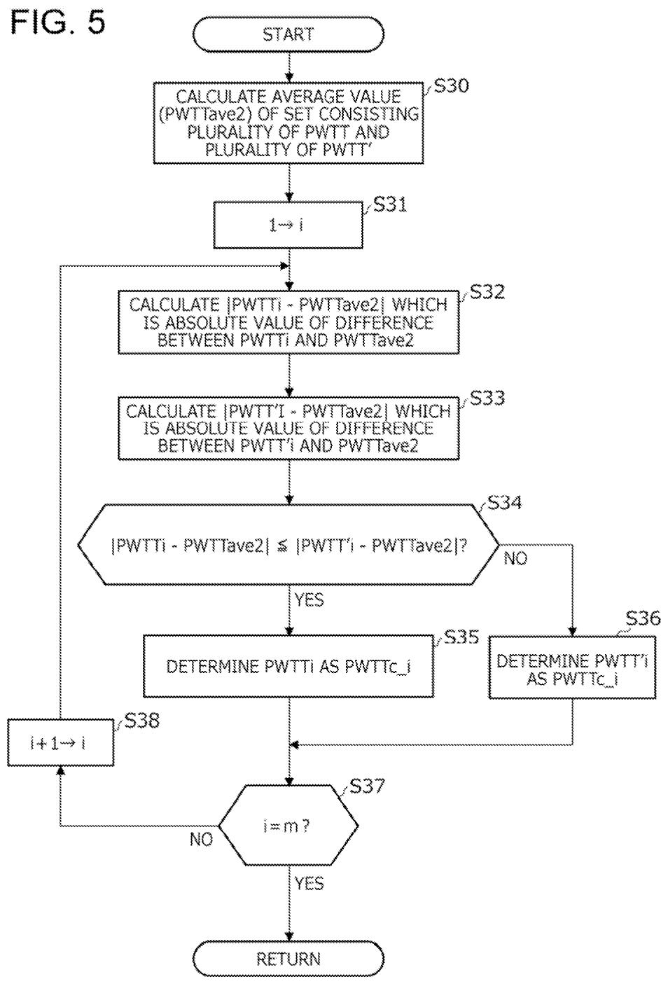

Next, the controller 2 determines a plurality of PWTT_{c} which are candidate values of the plurality of PWTTᵢ based on the plurality of PWTTᵢ and the plurality of PWTT′ᵢ (step S7). Here, an example of a calculation method of each of the plurality of PWTT′_{c} will be described with reference to FIG. 5. FIG. 5 is a flow chart for explaining an example of a process for determining each of the plurality of PWTT_{c} which are candidate values of the plurality of PWTT. Incidentally, assume that each of the m PWTT_{c} of the m PWTTᵢ is determined in the process shown in FIG. 5. In the following description, assume that the candidate value of the PWTTᵢ is denoted by PWTT_{c_i}. For example, a candidate value of a PWTTᵢ is denoted by PWTT_{c_1}.

As shown in FIG. 5, the controller 2 first calculates an average value PWTTₐᵥₑ₂ of a set consisting of the plurality of PWTTᵢ and the plurality of PWTT′ᵢ (step S30). For example, the PWTTₐᵥₑ₂ can be expressed by the following expression.

$$\begin{matrix}[ Math . 6 ] & \\ P W T {T}_{aνe2} = \frac{1}{2 m} {\sum }_{i = 1}^{m} {PWTT}_{i} + {{PWTT}^{′}}_{i} & ( 6 )\end{matrix}$$

Incidentally, the PWTTₐᵥₑ₂ may be the average value PWTTₐᵥₑ of the plurality of PWTTᵢ (i=1, 2, . . . m) or may be an average value of the plurality of PWTT′ᵢ. Further, the PWTTₐᵥₑ₂ may be an average value of a plurality of PWTTᵢ in the preceding time interval Tₙ₋₁.

Next, an initial value of i is set as 1 in a step S31. That is, in the process shown in FIG. 5, first, the PWTT_{c_1} which is the candidate value of the PWTT₁ is determined, and then PWTT_{c_2} which is a candidate value of a PWTT₂ is determined. Thus, the PWTT_{c_1} to a PWTT_{c_m} are determined by the process shown in FIG. 5.

Next, the controller 2 calculates [PWTTᵢ−PWTTₐᵥₑ₂] which is an absolute value of a difference between the PWTTᵢ and the PWTTₐᵥₑ₂ (step S32). Further, the controller 2 calculates [PWTT₁−PWTTₐᵥₑ₂] which is an absolute value of a difference between the PWTT′ᵢ and the PWTTₐᵥₑ₂ (step S33). Then, the controller 2 determines whether the absolute value of the difference between the PWTT′ᵢ and the PWTTₐᵥₑ₂ is equal to or larger than the absolute value of the difference between the PWTTᵢ and the PWTTₐᵥₑ₂ or not (step S34). When the determination of the step S34 results in YES, the controller 2 determines the PWTTᵢ as the PWTT_{c_i} which is a candidate value of the PWTTᵢ (step S35). On the other hand, when the determination of the step S34 results in NO, the controller 2 determines the PWTT′ᵢ as the PWTT_{c_i} which is the candidate value (step S36). Next, after the value of i is updated from 1 to 2 through steps S37 and S38, the process of the steps S32 to S36 is executed again. In this manner, a plurality of PWTT_{c} which are candidate values of a plurality of PWTT are determined.

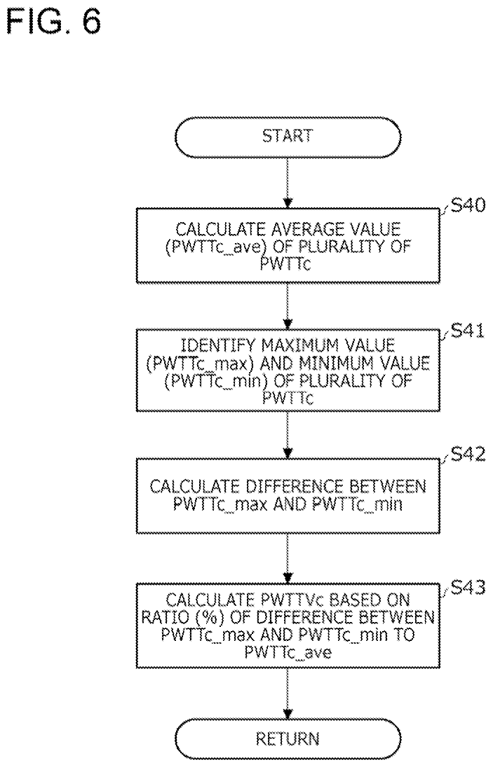

Return to FIG. 2. In a step S8, the controller 2 calculates a PWTT_{c} variation PWTTV_{c} in the time interval Tₙ. Here, an example of a calculation method of the PWTTV_{c} will be described with reference to FIG. 6. FIG. 6 is a flow chart for explaining an example of a process for calculating the PWTT_{c} variation PWTTV_{c}.

As shown in FIG. 6, the controller 2 first calculates an average value PWTT_{c_ave} of the plurality of PWTT_{c} (step S40). For example, the PWTT_{c_ave} can be expressed by the following expression. Here, assume that m PWTT_{c_i} (i=1, 2, . . . m) are present in the time interval Tₙ.

$$\begin{matrix}[ Math . 7 ] & \\ {PWTT}_{c\_ave} = \frac{1}{m} {Σ}_{i = 1}^{m} {PWTT}_{c\_ i} & ( 7 )\end{matrix}$$

Next, the controller 2 identifies a maximum value PWTT_{c_max} and a minimum value PWTT_{c_min} of the plurality of PWTT_{c} in the time interval Tₙ respectively (step S41). Then, the controller 2 calculates a difference between the maximum value PWTT_{c_max} and the minimum value PWTT_{c_min} (step S42). Next, in a step S43, the controller 2 calculates a PWTTV_{c} based on a ratio (%) of the difference between the maximum value PWTT_{c_max} and the minimum value PWTT_{c_min} to the average value PWTT_{c_ave} of the PWTT_{c}. For example, the PWTTV_{c} can be expressed by the following expression. In this manner, the PWTTV_{c} in the time interval Tₙ can be calculated.

[Math.8]

PWTTV_{c}=(PWTT_{c_max}−PWTT_{c_min})/PWTT_{c_ave}×100% (8)

Return to FIG. 2 again. In a step S9, the controller 2 determines whether the PWTTV_{c} in the time interval Tₙ satisfies a predetermined condition associated with the plurality of previously calculated PWTTV (specifically, the PWTTVₙ₋₁ to the PWTTVₙ₋ₚ) or not. Specifically, the controller 2 determines whether the PWTTV_{c} satisfies the following conditional expression or not. Here, the PWTTVₐᵥₑ is the average value of the plurality of previously calculated PWTTV defined by the expression (3).

[Math.9]

PWTTVₐᵥₑα≤PWTTV_{c}≤PWTTVₐᵥₑαα (9)

Thus, it is determined whether the PWTTV_{c} satisfies the predetermined condition which is relevant to the PWTTVₐᵥₑ and defined by the aforementioned expression (9) or not. When the determination of the step S9 results in YES, the controller 2 determines the plurality of calculated PWTT_{c} as normal values of the plurality of PWTT in the time interval Tₙ (step S10). In this case, the plurality of PWTT_{c} determined as the normal values are stored in the memory or the storage device 3. On the other hand, when the determination of the step S9 results in NO, the controller 2 determines the plurality of calculated PWTT_{c} as abnormal values of the plurality of PWTT in the time interval Tₙ (step S11). In this case, the plurality of PWTT_{c} determined as the abnormal values are deleted from the memory or the storage device 3. Thus, a series of processes shown in FIG. 2 are executed.

According to the present embodiment, when the PWTTV does not satisfy the predetermined condition associated with the plurality of previously calculated PWTTV (i.e. when the determination of the step S4 results in NO), the plurality of PWTT′ᵢ which are the corrected values of the plurality of PWTTᵢ are calculated based on the plurality of PWTTᵢ and RR intervals immediately previous thereto. Further, the plurality of PWTT_{c_i} which are the candidate values of the plurality of PWTT are determined based on the plurality of PWTTᵢ and the plurality of PWTT′ᵢ. In this manner, when it is determined that the calculated values of the plurality of PWTT are not correct, the plurality of PWTT are replaced by the plurality of PWTT_{c}. Accordingly, it is possible to further improve calculation accuracy of the plurality of PWTT.

In addition, when the PWTTV satisfies the predetermined condition (i.e. the determination of the step S4 results in YES), the calculated values of the plurality of PWTT are determined as normal values of the plurality of PWTT. On the other hand, when the PWTTV does not satisfy the predetermined condition (i.e. the determination of the step S4 results in NO), the calculated values of the plurality of PWTT are replaced by the plurality of PWTT_{c}. In this manner, it is possible to determine propriety of the calculated values of the plurality of PWTT according to whether the PWTTV satisfies the predetermined condition or not.

Further, when the PWTTV_{c} satisfies the predetermined condition (i.e. when the determination of the step S9 results in YES), the plurality of PWTT_{c} are determined as normal values of the plurality of PWTT. On the other hand, when the PWTTV_{c} does not satisfy the predetermined condition (i.e. when the determination of the step S9 results in NO), the plurality of PWTT_{c} are determined as abnormal values. In this manner, it is possible to determine propriety of the plurality of PWTT_{c} according to whether the PWTTV_{c} satisfies the predetermined condition or not.

In addition, in order to realize the processing apparatus 1 according to the present embodiment by software, the physiological information processing program may be incorporated into the storage device 3 or the ROM in advance. Alternatively, the physiological information processing program may be stored in a computer-readable storage medium such as a magnetic disk (e.g. an HDD or a floppy disk), an optical disk (e.g. a CD-ROM, a DVD-ROM or a Blu-ray (registered trademark) disk). an magneto-optical disk (e.g. an MO), a flash memory (e.g. an SD card, a USB memory or an SSD), or the like. In this case, the physiological information processing program stored in the storage medium may be incorporated into the storage device 3. Further, after the program incorporated into the storage device 3 is loaded onto the RAM, the processor may execute the program loaded onto the RAM. In this manner, the physiological information processing method according to the present embodiment is executed by the processing apparatus 1.

In addition, the physiological information processing program may be downloaded from a computer on the communication network through the network interface 4. Also in the case, the downloaded program may be incorporated into the storage device 3 in a similar manner or the same manner.

Although the embodiment of the present invention has been described above, the technical scope of the present invention should not be interpreted limitedly to the description of the present embodiment. It should be understood by those skilled in the art that the present embodiment is merely an example and various changes can be made on the embodiment within the scope of the invention described in CLAIMS. The technical scope of the present invention should be determined based on the scope of the invention described in CLAIMS and the scope of equivalents thereto.

This application is based on Japanese Patent Application No. 2018-166764 filed on Sep. 6, 2018, the entire contents of which are incorporated herein by reference.

1. A physiological information processing method executed by a computer, the method comprising:
acquiring electrocardiogram data of a subject;
acquiring pulse wave data of the subject;
calculating a plurality of RR intervals in a predetermined time interval based on the electrocardiogram data;
calculating a plurality of pulse wave transit times (PWTT) in the predetermined time interval based on the electrocardiogram data and the pulse wave data;
calculating a pulse wave transit time variation (PWTTV) in the predetermined time interval based on the plurality of PWTT in the predetermined time interval;
determining whether the PWTTV in the predetermined time interval satisfies a predetermined condition associated with a plurality of previously calculated PWTTV or not;
calculating corrected values (PWTT′) of the plurality of PWTT based on the plurality of PWTT and the plurality of RR intervals in a case where the PWTTV does not satisfy the predetermined condition; and
determining candidate values (PWTTc) of the plurality of PWTT based on the plurality of PWTT and the plurality of PWTT′.

2. The method according to claim 1, wherein
when the PWTTV satisfies the predetermined condition, the plurality of calculated PWTT are determined as normal values of the plurality of PWTT in the predetermined time interval.

3. The method according to claim 1, further comprising:
calculating a corrected value (PWTTVc) of the pulse transit time variation in the predetermined time interval based on the plurality of PWTTc; and
determining whether the PWTTVc satisfies the predetermined condition or not,
wherein:
when the PWTTVc satisfies the predetermined condition, the plurality of PWTTc are determined as normal values of the plurality of PWTT in the predetermined time interval, and
when the PWTTVc does not satisfy the predetermined condition, the plurality of PWTTc are determined as abnormal values of the plurality of PWTT in the predetermined time interval.

4. The method according to claim 1, wherein the calculating of the plurality of PWTT′ comprises:
adding a first RR interval calculated immediately before a first PWTT of the plurality of PWTT, to the first PWTT to thereby calculate a first PWTT′ of the plurality of PWTT′.

5. The method according to claim 1, wherein the calculating of the PWTTV comprises:
calculating an average value of the plurality of PWTT;
calculating a difference between a maximum value and a minimum value of the plurality of PWTT; and
calculating the PWTTV based on a ratio of the difference to the average value of the plurality of PWTT.

6. The method according to claim 1, wherein the predetermined condition is associated with a predetermined range set based on an average value of the plurality of previously calculated PWTTV.

7. The method according to claim 1, wherein the determining of the plurality of PWTTc comprises:
calculating an absolute value of a first difference between a first PWTT of the plurality of PWTT and a predetermined value;
calculating an absolute value of a second difference between a first PWTT′ of the plurality of PWTT′ which is a corrected value of the first PWTT and the predetermined value; and
determining the first PWTT′ as a first PWTTc of the plurality of PWTTc when the absolute value of the first difference is larger than the absolute value of the second difference.

8. The method according to claim 7, wherein the predetermined value is an average value of a set consisting of the plurality of PWTT and the plurality of PWTT′.

9. The method according to claim 3, wherein the calculating of the PWTTVc comprises:
calculating an average value of the plurality of PWTTc;
calculating a difference between a maximum value and a minimum value of the plurality of PWTTc; and
calculating the PWTTVc based on a ratio of the difference to the average value of the plurality of PWTTc.

10. The physiological information processing method according to claim 1, further comprising:
determining the predetermined time interval based on a respiration interval of the subject.

11-12. (canceled)

13. A physiological information processing apparatus comprising:
at least one processor; and
at least one memory storing a computer-readable instruction that when executed by the at least one processor, causes the physiological information processing apparatus to perform operations comprising:
acquiring electrocardiogram data of a subject;
acquiring pulse wave data of the subject;
calculating a plurality of RR intervals in a predetermined time interval based on the electrocardiogram data;
calculating a plurality of pulse wave transit times (PWTT) in the predetermined time interval based on the electrocardiogram data and the pulse wave data;
calculating a pulse wave transit time variation (PWTTV) in the predetermined time interval based on the plurality of PWTT in the predetermined time interval;
determining whether the PWTTV in the predetermined time interval satisfies a predetermined condition associated with a plurality of previously calculated PWTTV or not;
calculating corrected values (PWTT′) of the plurality of PWTT based on the plurality of PWTT and the plurality of RR intervals in a case where the PWTTV does not satisfy the predetermined condition; and
determining candidate values (PWTTc) of the plurality of PWTT based on the plurality of PWTT and the plurality of PWTT′.

14. The physiological information processing apparatus according to claim 13, wherein when the PWTTV satisfies the predetermined condition, the physiological information processing apparatus determines the plurality of calculated PWTT as normal values of the plurality of PWTT in the predetermined time interval.

15. The physiological information processing apparatus according to claim 13, wherein when executed by the at least one processor, the computer-readable instruction causes the physiological information processing apparatus to perform operations further comprising:
calculating a corrected value (PWTTVc) of the pulse transit time variation in the predetermined time interval based on the plurality of PWTTc;
determining whether the PWTTVc satisfies the predetermined condition or not;
determining the plurality of PWTTc as normal values of the plurality of PWTT in the predetermined time interval, when the PWTTVc satisfies the predetermined condition; and
determining the plurality of PWTTc as abnormal values of the plurality of PWTT in the predetermined time interval, when the PWTTVc does not satisfy the predetermined condition.

16. The physiological information processing apparatus according to claim 13, wherein when calculating the plurality of PWTT′, the apparatus adds a first RR interval calculated immediately before a first PWTT of the plurality of PWTT, to the first PWTT to thereby calculate a first PWTT′ of the plurality of PWTT′.

17. The physiological information processing apparatus according to claim 13, wherein when calculating the PWTTV, the apparatus:
calculates an average value of the plurality of PWTT;
calculates a difference between a maximum value and a minimum value of the plurality of PWTT; and
calculates the PWTTV based on a ratio of the difference to the average value of the plurality of PWTT.

18. The physiological information processing apparatus according to claim 13, wherein the predetermined condition is associated with a predetermined range set based on an average value of the plurality of previously calculated PWTTV.

19. The physiological information processing apparatus according to claim 13, wherein
when determining the plurality of PWTTc, the apparatus:
calculates an absolute value of a first difference between a first PWTT′ of the plurality of PWTT and a predetermined value;
calculates an absolute value of a second difference between a first PWTT′ of the plurality of PWTT′ which is a corrected value of the first PWTT and the predetermined value; and
determines the first PWTT′ as a first PWTTc of the plurality of PWTTc when the absolute value of the first difference is larger than the absolute value of the second difference.

20. The physiological information processing apparatus according to claim 19, wherein the predetermined value is an average value of a set consisting of the plurality of PWTT and the plurality of PWTT′.

21. The physiological information processing apparatus according to claim 15, wherein
when calculating the PWTTVc, the physiological information processing apparatus:
calculates an average value of the plurality of PWTTc;
calculates a difference between a maximum value and a minimum value of the plurality of PWTTc; and
calculates the PWTTVc based on a ratio of the difference to the average value of the plurality of PWTTc.

22. The physiological information processing apparatus according to claim 13, wherein when executed by the at least one processor, the computer-readable instruction causes the physiological information processing apparatus to perform operations further comprising:
determining the predetermined time interval based on a respiration interval of the subject.
